(19) United States
(12) Patent Application Publication
(10) Pub. No.: US 2023/0022328 A1
(43) Pub. Date: Jan. 26, 2023

(54) Method for Manufacturing a Fibrinogen Preparation

(71) Applicant: Biotest AG, Dreieich (DE)

(72) Inventors: Vera Ott, Langen (DE); Wolfgang Möller, Oberursel (DE); Oliver Maneg, Bad Homburg (DE)

(21) Appl. No.: 17/782,684

(22) PCT Filed: Dec. 8, 2020
(86) PCT No.: PCT/EP2020/085094

§ 371 Date: Jun. 6, 2022

(30) Foreign Application Priority Data
Dec. 10, 2019 (EP) 19214919.3

(51) Int. Cl. C07K 14/75; C07K 1/18; C07K 1/34; A61K 9/19; A61K 38/36

(52) U.S. Cl. CPC C07K 14/75; C07K 1/18; C07K 1/34; A61K 9/19; A61K 38/363

(57) ABSTRACT

A method for manufacturing a fibrinogen preparation from a fibrinogen containing source derived from blood plasma includes providing a liquid phase containing plasmatic fibrinogen; contacting the liquid phase with a cation exchange chromatography material under conditions resulting in binding of fibrinogen, wherein the liquid phase has a pH in the range of pH 5.6 to pH 7.0 which is near or above the pl of fibrinogen; optionally washing unbound compounds from the cation exchange chromatography material; and eluting the fibrinogen from the cation exchange material. The method is also suitable for reduction of von-Willebrand-factor.

The invention is concerned with manufacturing a fibrinogen preparation from a fibrinogen containing source derived from blood plasma.

Fibrinogen is the main structural protein in blood responsible for the formation of clots. It is very important for treating haemostatic disorders.

An important source for fibrinogen is its isolation from blood plasma, especially human blood plasma. During this purification, other components of the blood plasma or the production process have to be removed with the aim of a specific and pure coagulation blood product. These other components are usually other plasma proteins, especially von-Willebrand-factor (vWF).

Purification of fibrinogen from plasma is usually accomplished using precipitation techniques combined with subsequent chromatographic techniques and virus inactivation steps like Solvent/Detergent (S/D) and/or virus removal steps like filtration. It is therefore not only important to remove the other blood plasma proteins, but also reagents or additives used in previous process steps.

WO 00/17234 A1/EP 1 115 742 B1 discloses the purification of recombinant fibrinogen from milk from transgenic animals using cation exchange chromatography (CEX) to remove the milk protein casein from the preparation.

WO 98/38219 A1 discloses the purification of vWF (isoelectric point of 5.5 to 6) using a negatively charged gel matrix of a cation exchange chromatography. vWF binds to the cation exchanger at a low salt concentration and is eluted in a buffer having a pH in the range of 5.0 to 8.5.

WO 91/01808 A1 discloses a selective elimination of LDL, fibrinogen and/or urea from aqueous liquids like whole blood, plasma or serum. The method uses an adsorption material based on Fractogel® material (Merck, Germany) modified with polymeric chains of monomers containing sulfonate groups forming a graft copolymer (tentacle cation exchange material). The specific adsorption properties of the cation exchange material are dependent from the presence of the tentacle-like ligand structure.

EP 2 267 025 A2 describes the use of CEX for purification of fibrinogen. The fibrinogen does not bind to the CEX matrix.

It is object of the present invention to provide a method for manufacturing a fibrinogen preparation from a fibrinogen-containing source derived from blood plasma, which allows an easy and efficient purification of fibrinogen preferably with improved properties.

The problem is solved by the invention as claimed in the independent claims. The aim is achieved by a method for manufacturing a fibrinogen preparation from a fibrinogen containing source derived from blood plasma comprising the following steps:
a) Providing a liquid phase containing plasmatic fibrinogen;
b) Contacting the liquid phase with a cation exchange chromatography material under conditions resulting in binding of fibrinogen, wherein the liquid phase has a pH in the range of pH 5.6 to pH 7.0 which is near or above the pl of fibrinogen;
c) Optionally washing unbound compounds from the cation exchange chromatography material;
d) Eluting the fibrinogen from the cation exchange material.

The object of the invention is also achieved by a method as described in the following. In what follows, individual steps of a method will be described in more detail. The steps do not necessarily have to be performed in the order given in the text. In addition, further steps not explicitly stated may be part of the method.

As used herein the term “fibrinogen” refers to the main structural protein responsible for the formation of clots as present in blood plasma and preferably refers to the whole glycoprotein form of fibrinogen. Preferably, it refers to plasmatic fibrinogen, i.e. a fibrinogen, which is derived from plasma. More preferably, it is a human plasmatic fibrinogen.

The pl or the isoelectric point (IEP) of a protein is the pH value at which the protein carries no net charge. At pH values above the pl, the protein has a net negative charge and at pH values below the pl, the protein has a net positive charge. As used herein, the pl of fibrinogen is pH 5.5 as mentioned e.g. in WO 00/17234 A1. Other sources report a pl of fibrinogen in the range of about pH 5.5 up to 6.0, depending on the clipping of polar residues in the clotting response by thrombin (Guo et al. nature nanotechnology 2016, 11, 817-824)

As used herein, “cation exchange chromatography (CEX) material” is a solid phase, which contains negatively charged groups. Proteins are separated based on the interaction between negatively charged groups in the resin and positively charged groups on a protein. The strength of the interaction is also depending on the ionic strength (i.e. conductivity) of the buffer. Elution is generally achieved by increasing the ionic strength of the buffer to compete with the protein for the charged sites of the cation exchange material. Changing the pH and thereby altering the charge of the protein is another way to achieve elution of a protein. The change in conductivity and/or pH may be gradual or stepwise.

The charge of the CEX material may be provided by attaching one or more charged ligands to the solid phase, e.g. by covalent linking. Preferred CEX materials in the context of the present invention are strong CEX materials. These are materials, which maintain a negative charge on the solid phase over a wide pH range. These usually incorporate sulfonic acids derivatives as functional groups, like sulfoethyl, sulfopropyl, sulfobutyl or sulfoisobutyl groups (e. g. sulfonates, S-type or sulfopropyl groups, SP-types).

Commercially available cation exchange materials include carboxy-methyl-cellulose, BAKERBOND ABX™, sulphopropyl (SP) immobilized on agarose (e. g. SP-SEPHAROSE FAST FLOW™, SP-SEPHAROSE FAST FLOW XL™ or SP-SEPHAROSE HIGH PERFORMANCE™, from GE Healthcare), CAPTO S™ (GE Healthcare), sulphonyl immobilized on agarose (e.g. S-SEPHAROSE FAST FLOW™, from GE Healthcare), and SUPER SP™ (Tosoh Biosciences). A preferred cation exchange material herein comprises cross-linked poly(styrene-divinylbenzene) flow-through particles (solid phase) coated with a polyhydroxylated polymer functionalized with sulfopropyl groups (for example, POROS™ 50 HS chromatography resin, from Thermo Fisher Scientific) or methacrylic copolymer with sulfonic acid groups (for example, Macro-Prep® High S, from Bio-Rad). Especially preferred CEX materials comprise pores with an average pore size of 50 nm, preferably of 100 nm, e.g. an average pore size of 160 nm.

According to prior art processes, a person skilled in the art uses a CEX substrate in such a way that the pH of the medium in which the separation is carried out is below the pl of the protein of interest, resulting in a binding of the protein of interest to the CEX substrate.

By “solid phase” is meant a non-aqueous matrix to which one or more charged ligands can adhere. The solid phase may be a purification column (including, without limitation, expanded bed and packed bed columns), a discontinuous phase of discrete particles, a membrane, or filter etc. Examples of materials for forming the solid phase include polysaccharides (such as agarose and cellulose) and other mechanically stable matrices such as silica (e. g. controlled pore glass), poly(styrene-divinylbenzene), methacrylate copolymer, polyacrylamide, ceramic particles and derivatives of any of the above, preferred are poly(styrene-divinylbenzene) and methacrylate copolymer.

The term “liquid phase containing plasmatic fibrinogen” herein refers to the composition loaded onto the cation exchange material. Preferably, the cation exchange material is equilibrated with an equilibration buffer prior to loading the composition, which is to be purified.

A “buffer” is a solution that resists changes in pH by the action of its acid-base conjugate components. Various buffers can be employed depending on the desired pH of the buffer.

An “equilibration buffer” is a buffer that is used to equilibrate the cation exchange material, prior to loading the liquid phase containing fibrinogen onto the cation exchange material. The composition of the equilibration buffer depends on the CEX material used.

The term “wash buffer” as used herein refers to the buffer that is passed over the cation exchange material following loading of a composition and prior to elution of fibrinogen bound to the CEX material. The wash buffer may serve to remove one or more contaminants from the cation exchange material, without substantial elution of the bound fibrinogen. More than one wash buffer can be used prior eluting the bound fibrinogen.

“Elution buffer” is used to elute fibrinogen from the solid phase. Herein, the elution buffer has a substantially increased conductivity and/or increased pH relative to that of the last wash buffer, such that the fibrinogen is eluted from the cation exchange material. Preferably, the conductivity and/or pH of the elution buffer is substantially greater than that of the liquid phase containing fibrinogen and of each of the preceding buffers, namely of the equilibration buffer and all wash buffers used. By “substantially greater conductivity” is meant, for example, that the buffer has a conductivity, which is at least 2, 3, 4, 5, 6, 7, 8, 9, 10 conductivity units (mS/cm) greater than that of the composition or buffer to which it is being compared. By “substantially greater pH” is meant, for example, that the buffer has a pH, which is at least 0.2, 0.3, 0.4, 0.5 pH units greater than that of the composition or buffer to which it is being compared. The conditions for elution depend on the CEX material used.

A “regeneration buffer” may be used to regenerate the cation exchange material such that it can be re-used. The regeneration buffer has a conductivity and/or pH as required to remove substantially all contaminants and possibly remaining fibrinogen from the cation exchange material.

The term “conductivity” refers to the ability of an aqueous solution to conduct an electric current between two electrodes. In solution, the current flows by ion transport. Therefore, with an increasing amount of ions present in the aqueous solution, the solution will have a higher conductivity. The basic unit of measure for conductivity is the Siemens per meter (S/m) usually measured in mS/cm, and can be measured using a conductivity meter. Since electrolytic conductivity is the capacity of ions in a solution to carry electrical current, the conductivity of a solution may be altered by changing the concentration of ions therein. For example, the concentration of a buffering agent and/or the concentration of a salt (e.g. sodium chloride, sodium acetate, or potassium chloride) in the solution may be altered in order to achieve the desired conductivity. Preferably, the salt concentration of the various buffers is modified to achieve the desired conductivity. In addition, salts with organic cations may contribute to the conductivity, preferably cationic amino acids, preferably arginine, especially when they are present as hydrochlorides.

The term “fibrinogen containing source derived from blood plasma” defines that the source of the fibrinogen is blood plasma or plasma fractions, preferably human blood plasma. Particularly, it is not a recombinant or synthetic fibrinogen. More particularly, it is not manufactured by recombinant production in other organisms or liquids. The plasmatic source also defines the possibly relevant contaminants present, like Albumin, Fibronectin, IgG, vWF or fibrinopeptide A.

The method for manufacturing a fibrinogen preparation according to the inventive approach allows purification of a fibrinogen product with an improved purity and activity profile. The method is especially useful for separation of vWF from the fibrinogen preparation, thereby reducing the vWF content within the resulting composition. Preferably, both fibrinogen and vWF bind to the cation exchange chromatography material in step b) wherein the liquid phase has a pH in the range of pH 5.6 to 7.0, preferably pH 6.0 to 6.9, more preferably pH 6.3 to 6.9. When eluting fibrinogen from the material according to step d) fibrinogen is selectively eluted wherein vWF remains on the cation exchange material. E.g. elution is done at pH 7.0 which allows selective elution of fibrinogen wherein vWF still remains bound to the chromatography matrix.

Generally, according to the methodical theory of cation exchange chromatography fibrinogen should not bind to a cation exchange chromatography material at a pH that lies above the pl of the protein of interest. Accordingly, e.g. EP 2 267 025 A2 describes the application of a cation exchange chromatography step within a process of purification of fibrinogen, wherein fibrinogen does not bind to the matrix. Surprisingly, the inventors have found that fibrinogen binds to the chromatography material under the conditions as used herein, especially at a pH value, which lies above the pl value of fibrinogen. In addition, also vWF binds to the chromatography material, although also vWF shows an isoelectric point, which is very similar to the isoelectric point of fibrinogen. According to WO 98/38219 A1 the isoeletric point of vWF is in the range of 5.5 to 6. Therefore, it is surprising, that firstly both proteins bind to the material and secondly both proteins and especially fibrinogen may be selectively eluted, although their isoelectric characteristics are very similar. The inventors have found that under the specific conditions applied vWF binds slightly stronger than fibrinogen. Therefore, according to the inventive method, it is possible to selectively elute fibrinogen from the column, and this chromatography step may be applied to effectively reduce the amount of vWF within the fibrinogen preparation. Thus, the invention also relates to a method for the reduction of the vWF content within a composition comprising fibrinogen by the use of a cation exchange chromatography as described herein.

Thus, in a preferred embodiment of the invention the liquid phase containing the fibrinogen further contains vWF. The method may be used for a liquid phase wherein vWF is present in an amount of at least 0.3 U/mg total protein (e.g. assessment of VWF level by means of VWF antigen), e.g. of at least 0.4 U/mg total protein or more, wherein the total protein is measured using OD 280. In preferred embodiments the vWF content of the liquid phase is about 0.4-1.0 U/mg total protein. The vWF content depends on the source of the fibrinogen and the previous purification steps. Thus, the vWF content may be even higher.

In a preferred embodiment, the liquid phase, which is to be loaded onto the CEX material, is an already partly purified fibrinogen preparation. The partly purified fibrinogen preparation may comprise 90% or more fibrinogen, preferably 95% fibrinogen or even more. In a preferred embodiment, the liquid phase does comprise other proteins except fibrinogen, preferably other plasma proteins, in an amount of less than 10%, preferably less than 5%. The content of vWF in the liquid phase which is to be loaded onto the CEX material may be e.g. 0.4-1.0 U/mg total protein. Preferably, the vWF content of the load material is at least 0.5 U/mg total protein, preferably 0.5 to 1.0 U/mg total protein.

Moreover, the method is especially useful for reducing the amount of prions in the fibrinogen preparation. The inventors were able to demonstrate that prions are effectively removed to below the limit of detection by the cation exchange chromatography step as described. To show this effect Hamster prions as a well-established test model for variant Creutzfeldt-Jacob disease have been used in spiking experiments. Thus, the invention also relates to a method for removal or elimination of prions within a composition comprising fibrinogen by the use of a cation exchange chromatography as described herein.

The liquid phase containing the fibrinogen is preferably an aqueous phase. The liquid phase can further comprise one or more detergents like polysorbate-80 and/or solvents like tributyl phosphate (TnBP). Such detergents and solvents can be present due to a previous S/D-treatment for virus removal. It is a particular advantage of the inventive method that the method also allows the removal of such other unwanted compounds within the preparation, which may derive from previous manufacturing steps, like the removal of detergents and/or solvents from former virus inactivation steps like from a preceding S/D treatment.

In a preferred embodiment, the fibrinogen containing source is subjected to a virus inactivating process, for example a solvent detergent process (S/D-treatment).

In a preferred embodiment, the liquid phase is prepared by resuspending the precipitate of a glycine precipitation.

The liquid phase is brought in contact with a cation exchange chromatography material under conditions resulting in binding of fibrinogen, wherein the liquid phase has a pH of between 5.6 and 7.0. It may be necessary to further adjust the pH and/or conductivity of the liquid phase to the corresponding conditions prior to loading. The liquid phase is then loaded onto the cation exchange chromatography material.

In an especially preferred embodiment, the liquid phase has a pH in the range of 6.0 to 6.9, more preferably 6.3 to 6.9, even more preferably 6.4 to 6.8, most preferably 6.5 to 6.7, when contacting it with the cation exchange chromatography material.

The ionic strength of the liquid phase is preferably in the range of 5 to 15 mS/cm, more preferably 7 to 11 mS/cm, especially 9.0+/−1.5 mS/cm, preferably 9.0+/−1.0 mS/cm, when contacting it with the cation exchange chromatography material.

In a preferred embodiment, the liquid phase has a pH in the range of 5.6 to 7.0 and an ionic strength in the range of 5 to 15 mS/cm, more preferably a pH in the range of 6.0 to 6.9 and an ionic strength in the range of 5 to 15 mS/cm, even more preferably a pH in the range of 6.3 to 6.9 and an ionic strength in the range of 7 to 11 mS/cm, most preferably a pH in the range of 6.4 to 6.8 and an ionic strength in the range of 9.0+/−1.5 mS/cm, more preferably a pH in the range of 6.4 to 6.8 and an ionic strength in the range of 9.0+/−1.0 mS/cm or even a pH in the range of 6.5 to 6.7 and an ionic strength in the range of 9.0+/−1.0 mS/cm.

In a preferred embodiment, the pH of the liquid phase is stabilized by using a buffer system. This may be citrate, phosphate or acetate buffers. Preferred buffers are citrate buffers, more preferred tri-sodium citrate buffers.

In a preferred embodiment, the concentration of buffer is below 50 mM, more preferably below 30 mM, depending on the ionic strength to be obtained. The concentration of buffer is preferably above 5 mM, more preferably above 10 mM. In a preferred embodiment 15 mM concentration of buffer substance.

If required the ionic strength may be adjusted by adding one or more salts, preferably a halogenid salt soluble in the liquid phase, more preferably sodium chloride.

Preferably, the protein load is not more than 22 g/l cation exchange chromatography material, preferably not more than 21 g/l, most preferably not more than 20 g/l. In preferred embodiments the protein load is from 5 to 22, preferably from 10 to 21 g/l, even more preferred from 10 to 20 g/l.

The process of cation exchange chromatography is preferably run at a temperature from 16° C. to 28° C., more preferably 18° C. to 26° C., more preferably at 22° C.+/−4° C.

The cation exchange chromatography material is preferably a strong CEX material.

The CEX material is preferably a material comprising particles with pores. Preferably, the CEX material is a macroporous material, especially a polymeric macroporous material. In a preferred embodiment, the CEX material comprises particles having a pore diameter range of 50 nm to 1000 nm, more preferably of 100 to 1000 nm. Preferably, the average pore size is 160 nm. In especially preferred embodiments, the chromatography material comprises small diffusive pores and additionally relatively large throughpores allowing a small percentage of convective flow through the particles such that diffusion is no longer limiting. Preferably in such a material the chromatography material comprises particles with large pores with a pore diameter of 200 nm to 1000 nm and small pores with a diameter of 5 to 30 nm, preferably large pores with a diameter of 250 nm to 600 nm and small pores with a diameter of 5 to 20 nm (measured as disclosed in Pirrung S. M. et al. Biotechnol. Prog. 2018, 34(4), 1006-1018).

In a preferred embodiment, the CEX material comprises particles with an average particle size of 30 to 100 μm, preferably 30 to 70 μm, more preferably about 50 μm.

In a preferred embodiment, the CEX material contains functional groups, especially sulfonate functional groups (—SO₃⁻). Preferably, the sulfonate groups may be attached to a linear or branched alkyl group of 2 to 6 carbon atoms, preferably 2 to 4 carbon atoms, more preferably ethyl, propyl, butyl groups, more preferably ethyl, n-propyl and isobutyl, even more preferably sulfopropyl (which corresponds preferably to n-propyl). In a preferred embodiment, the sulfonate groups on the cation exchange material are connected to the polymer chain of the CEX material.

In another preferred embodiment, the CEX material is not a tentacle cation exchange material (e.g. Fractogel® by Merck Millipore). These are graft polymers with polymeric side chains of monomers comprising SO³⁻-groups. Such CEX materials tend to bind fibrinogen and similar proteins too strong in the context of the purpose of the present invention.

In a preferred embodiment, the material comprises a polyhydroxyl surface coating with sulfopropyl group. The resin backbone preferably is made of crosslinked polystyrenedivinylbenzene, wherein the sulfonate functional groups are linked as sulfopropyl via a polyhydroxyl surface. One example of a preferred CEX material is POROS™ chromatography resin; even more preferred POROS™ 50 HS (ThermoFisher, USA). This material also comprises particles with large pores and small pores as defined above.

In another embodiment of the invention, the CEX material is a methacrylate copolymer with sulfonate functional groups. In this embodiment, the average particle size may be 50 μm and the average pore diameter may be 100 nm. One example of a preferred CEX material according to this embodiment is Macro-Prep®, even more preferred Macro-Prep® High S (BioRad, USA).

In a preferred embodiment, the CEX material is equilibrated prior adding the liquid phase containing fibrinogen to the CEX material. This is performed by using an equilibration buffer. Preferably, the equilibration buffer has a pH range of 5.6 and 7.0, more preferably 6.0 and 6.9, even more preferable 6.3 and 6.9, more preferably 6.4 to 6.8, even more preferably 6.5 to 6.7.

The ionic strength of the equilibration buffer is preferably 5 to 15 mS/cm, more preferably 7 to 12 mS/cm, especially 9.0+/−1.5 mS/cm or 9.0+/−1 mS/cm.

In a preferred embodiment, the equilibration buffer has a pH in the range of 5.6 to 7.0 and an ionic strength in the range of 5 to 15 mS/cm, more preferably a pH in the range of 6.0 to 6.9 and an ionic strength in the range of 5 to 15 mS/cm, even more preferably a pH in the range of 6.3 to 6.9 and an ionic strength in the range of 7 to 11 mS/cm, most preferably a pH in the range of 6.4 to 6.8 and an ionic strength in the range of 9.0+/−1.5 mS/cm, more preferably a pH in the range of 6.4 to 6.8 and an ionic strength in the range of 9.0+/−1.0 mS/cm or even a pH in the range of 6.5 to 6.7 and an ionic strength in the range of 9.0+/−1.0 mS/cm.

In a preferred embodiment, the pH of the equilibration buffer is stabilized by using a buffer system. Preferred buffer systems are phosphate, acetate or citrate buffers, preferably citrate buffers, more preferably based on tri-sodium-citrate.

In a preferred embodiment, the concentration of the buffer is below 50 mM, more preferably below 30 mM, depending on the ionic strength to be obtained. The concentration of buffer is preferably above 5 mM, more preferably above 10 mM. In a preferred embodiment it is 15 mM of buffer component.

In a preferred embodiment, the equilibration buffer comprises a salt, preferably sodium chloride. The concentration of the salt is preferably below 100 mM, more preferably below 80 mM, especially below 70 mM. As lower range the concentration of salt is preferably above 40 mM, more preferably above 50 mM and even more preferably above 60 mM, each also in combination with the previous upper limits, preferably between 40 mM and 80 mM, more preferably between 60 and 80 mM, more preferably 65 mM.+−/1-2 mM. The amounts of buffer system and salt are adjusted to obtain the preferred ionic strength of the equilibration buffer.

The equilibration is preferably performed by rinsing the CEX material with at least 2 column volumes of equilibration buffer.

After loading the liquid phase onto the CEX material according to step b) of the method according to the invention as a next optional step the unbound compounds are washed from the CEX material. For such a step, the CEX material is washed with a wash buffer. Preferably, the wash buffer has a pH in the range of 5.6 to 7.0, more preferably 6.0 to 6.9, even more preferable 6.3 to 6.9, even more preferably 6.4 to 6.8, most preferably 6.5 to 6.7.

The ionic strength of the wash buffer is preferably 5 to 15 mS/cm, more preferably 7 to 11 mS/cm, especially 9.0+/−1.5 mS/cm or 9.0+/−1.0 mS/cm.

In a preferred embodiment, the wash buffer has a pH in the range of 5.6 to 7.0 and an ionic strength in the range of 5 to 15 mS/cm, more preferably a pH in the range of 6.0 to 6.9 and an ionic strength in the range of 5 to 15 mS/cm, even more preferably a pH in the range of 6.3 to 6.9 and an ionic strength in the range of 7 to 11 mS/cm, most preferably a pH in the range of 6.4 to 6.8 and an ionic strength in the range of 9.0+/−1.5 mS/cm, more preferably a pH in the range of 6.4 to 6.8 and an ionic strength in the range of 9.0+/−1.0 mS/cm or even a pH in the range of 6.5 to 6.7 and an ionic strength in the range of 9.0+/−1.0 mS/cm.

In a preferred embodiment the pH of the wash buffer is stabilized by using a buffer system. Preferred buffer systems are phosphate, acetate or citrate buffers, preferably citrate buffers, more preferably based on tri-sodium-citrate.

In a preferred embodiment, the concentration of the wash buffer system is below 50 mM, more preferably below 30 mM, depending on the ionic strength to be obtained. The concentration of buffer is then above 5 mM, preferably above 10 mM, in a preferred embodiment 15 mM of buffer component.

In a preferred embodiment, the wash buffer comprises a salt, preferably a chloride salt, more preferably sodium chloride. The concentration of the salt is preferably below 100 mM, more preferably below 80 mM, especially below 70 mM. As lower range the concentration of salt is preferably above 40 mM, more preferably above 50 mM and even more preferably above 60 mM, each also in combination with the previous upper limits, preferably between 40 mM and 80 mM, more preferably between 60 and 80 mM, more preferably 65 mM+/−1-2 mM. The amounts of buffer system and salt are adjusted to obtain a wash buffer of the preferred ionic strength.

After loading, in a preferred embodiment, the CEX material is washed with at least 1 column volumes of wash buffer, preferably at least 2 column volumes of wash buffer.

In a preferred embodiment of the invention, the equilibration buffer and wash buffer have at least the same pH and/or ionic strength, more preferably the same pH and the same ionic strength. In an even more preferred embodiment, both buffers have the same composition.

After loading and preferably washing according to steps b) and c) of the method according to the invention in the next step the fibrinogen is eluted from the cation exchange material according to step d) of the method according to the invention. For this step, an elution buffer is passed over the CEX material. The elution buffer has an increased conductivity and/or increased pH relative to that of the last wash buffer or the liquid phase if no wash buffer is used, such that the fibrinogen is selectively eluted from the CEX material, while preferably vWF remains on the column. The conditions may depend on the CEX material used.

Preferably, the elution buffer has a pH at least 0.2; 0.3, 0.4 or 0.5 units above the conditions used for binding the fibrinogen to the CEX material. In a preferred embodiment, the pH is 0.2 to 1 pH units above the binding conditions, especially 0.2 to 0.5 units. In a preferred embodiment, the pH of the elution buffer is 7.0+/−0.1.

The ionic strength of the elution buffer is preferably at least 2, 3, 4, 5 mS/cm above the conditions used for binding the fibrinogen to the CEX material, more preferably at least 5 mS/cm above, even more preferably 5 to 15 mS/cm above, especially 10+/−1.5 mS/cm above the conditions used for binding the fibrinogen. In a preferred embodiment, the ionic strength is at least 1.5 to 2.5 times the ionic strength of the conditions used for binding, more preferably 1.8 to 2.2 times. In a preferred embodiment, the ionic strength is 19.5+/−1.5 mS/cm. All values refer to values at 20° C.

In a preferred embodiment, the elution buffer has a pH at least 0.2, 0.3 0.4 or 0.5 units above the conditions used for binding the fibrinogen to the CEX material and an ionic strength of more than at least 5 mS/cm above the conditions used for binding the fibrinogen to the CEX material, preferably a pH of 0.2 to 1 pH units above and 5 to 15 mS/cm, even more preferably a pH of 0.2 to 0.5 units above and 10+/−1.5 mS/cm above the conditions used for binding the fibrinogen.

In a preferred embodiment, the pH of the elution buffer is stabilized by using a buffer system. Preferred buffers are phosphate, acetate or citrate buffers, preferably citrate buffers, more preferably based on tri-sodium-citrate.

In a preferred embodiment, the concentration of the elution buffer system is below 30 mM, more preferably below 10 mM, also depending on the ionic strength to be obtained. The concentration of buffer is preferably between 5 mM and 30 mM, more preferably between 5 mM and 10 mM. In preferred embodiments, the buffer concentration is 7.5 mM.

In a preferred embodiment, the elution buffer comprises a salt, preferably sodium chloride. The concentration of the salt of the elution buffer is preferably above 100 mM, more preferably above 110 mM, especially above 120 mM, most preferably 150 mM. The concentration is preferably below 350 mM, more preferably below 250 mM. The amounts of buffer system and salt are adjusted to obtain the preferred ionic strength of the equilibration buffer.

In a preferred embodiment, the elution buffer comprises one or more drug formulation compound, e.g. amino acids, e.g. glycine, histidine, alanine, arginine, preferably cationic amino acids, preferably arginine. The amino acids can be added as hydrochlorides. In a preferred embodiment, the elution buffer comprises more than 50 mM of the drug formulation compound. Especially if the drug formulation compound contributes to the ionic strength, the concentration of salt in the elution buffer can be reduced. Therefore, it is especially preferred to use e.g. 50 to 100 mM drug formulation compound, preferably 70 to 80 mM, more preferably 75+/−2 mM drug formulation compound, preferably in combination with 100 to 200 mM sodium chloride, preferably 150+/−5 mM sodium chloride, within the elution buffer in order to provide sufficient ionic strength for elution of fibrinogen from the column material. Thereby, excessive salt concentrations are avoided which may be disadvantageous for the final drug product. Moreover, by using ingredients of the drug formulation for the purposes of elution, further buffer exchanging steps may be avoided.

In a preferred embodiment, the elution buffer comprises 50 to 100 mM arginine (preferably added as hydrochloride) and 100 to 200 mM sodium chloride, preferably 70 to 80 mM arginine and 100 to 200 mM sodium chloride, even more preferably 75+/−2 mM arginine and 150+/−5 mM sodium chloride.

The elution is performed until the fibrinogen is eluted from the column. This can be detected by measuring the UV-absorption.

If the inventive process is used as one of the final steps of a purification process a diafiltration step to remove excessive salt or other unwanted compounds can be omitted. Using an ultrafiltration step for concentration of the protein content may be sufficient before preparation of the drug substance composition from the product of the inventive process.

After elution of fibrinogen, the column may be regenerated using a regeneration buffer. During this regeneration step, vWF may be stripped of the column. The regeneration buffer may have a pH of 5.5 to 7.5, preferably 6.0 to 7.0, more preferably 6.5 to 6.7. The ionic strength is preferably above 50 mS/cm, more preferably above 100 mS/cm. In a preferred embodiment, the ionic strength is between 50 and 150 mS/cm, more preferably between 100 mS/cm and 130 mS/cm. The regeneration buffer may comprise, e.g., 1.5 M NaCl (high salt).

In a preferred embodiment, the material of the column is capable to be further cleaned using sodium hydroxide (NaOH) solutions, preferably with a concentration of at least 0.1 M, more preferably with a concentration of 0.5 M to 1.5 M sodium hydroxide, most preferably 1 M sodium hydroxide. After cleaning, the chromatography material may be stored in e.g. 0.1 M NaOH prior to further use.

The inventive process is especially useful to reduce the amount of vWF in the composition, especially in a composition derived from blood plasma. This is especially surprising, since vWF and fibrinogen have similar pl characteristics. Nevertheless, as it has been shown by the inventors, it is possible to selectively elute fibrinogen from the cation exchange material wherein vWF remains on the column. Thus, the inventive method is able to separate vWF from fibrinogen. Advantageously, the amount of vWF (in U/mg total protein) may be reduced by a factor of at least 2, preferably at least 3, more preferably up to 4 or even more, via the step of cation exchange chromatography according to the method of the invention, wherein of course the factor directly depends from the vWF content of the loading material. In preferred embodiments the amount of vWF in the eluted fibrinogen fraction is reduced to less than 0.5 U/mg total protein, more preferably to less than 0.4 U/mg, more preferably to less than 0.3 U/mg, even more preferably less than 0.2 U/mg. Typically, the eluted fibrinogen fraction from the cation exchange step may comprise a vWF content of about 0.1-0.3 U/mg total protein. The potential of the cation exchange chromatography step for reducing the vWF content in the fibrinogen fraction was also tested with vWF-spiked test material. The inventors were able to demonstrate that the depletion factor of vWF for the cation exchange chromatography step with spiked test material may be even 10 or higher.

The eluted fractions containing fibrinogen are preferably further concentrated and/or diluted to obtain a fibrinogen preparation with a defined fibrinogen content.

In a preferred embodiment, the eluted fractions containing fibrinogen comprise less than 0.01 mg/mL Polysorbat 80 and less than 0.8 μg/mL TnBP.

In a preferred embodiment, the method according to the invention further comprises a step e) of formulating the fibrinogen into a pharmaceutical composition.

The use of the cation exchange chromatography step within a purification protocol for manufacturing of fibrinogen from plasma has several advantages. Firstly, the cation exchange step is able to effectively reduce the amount of vWF within the fibrinogen product. Secondly, it is possible to already integrate further drug product ingredients like arginine and/or other formulation buffer ingredients in the elution buffer of the cation exchange chromatography, thereby avoiding further buffer exchange steps like diafiltration, which are costly and laborious. Thirdly, the cation exchange chromatography step is very effective in reducing or eliminating prions, thereby avoiding further extra steps for reducing or eliminating prions. A fourth advantage of the cation exchange chromatography step is the highly efficient removal of solvents/detergents such as Polysorbat 80 and TnBP. In sum, the manufacturing process including a cation exchange chromatography step as suggested by the inventors is a very effective and economical efficient method for the manufacturing of fibrinogen. The manufacturing process includes comparatively few and comparatively easy to handle process steps.

As already mentioned, the liquid phase to be loaded onto the cation exchange material may be an already partly purified fibrinogen preparation. Therefore, the inventive method for manufacturing a fibrinogen preparation may comprise preceding and/or successive purification and manufacturing steps, preferably including one or more virus inactivation steps. In an especially preferred embodiment, the manufacturing method further comprises at least one of the following steps:
using cryoprecipitate of human plasma as starting material;
Al(OH)₃ adsorption;
S/D treatment;
anion exchange chromatography and using the flow-through for further purification of fibrinogen;
glycine precipitation;
UV-C treatment;
ultrafiltration;
lyophilisation;
dry heat treatment.

In preferred embodiments, the complete manufacturing process starting with plasma includes the steps of an anion exchange chromatography, a glycine precipitation and a cation exchange chromatography as described herein. Preferably, further steps of virus inactivation respectively virus depletion are performed, like S/D treatment and/or UV-C treatment and/or dry heat treatment. By the step of anion exchange chromatography, a depletion factor for vWF of about 1 to 2 may be achieved. By the step of glycine precipitation, a depletion factor for vWF of about 4 to 5 may be achieved. By the step of cation exchange chromatography, a depletion factor for vWF of at least 3 may be achieved. For the complete manufacturing process an overall depletion factor for vWF of about 20 or even more may be achieved compared to the dissolved cryoprecipiate.

Preferably, the specific activity of the purified fibrinogen of the final product is at least 95% clottable protein activity as compared to total protein, preferably 98%±0.8 (measured activity by clottable protein and OD 280).

In an especially preferred embodiment, the manufacturing method comprises all of the above mentioned steps, wherein the step of cation exchange chromatography as described herein is performed between said UV-C treatment and said ultrafiltration. Preferably, the other steps are performed in the order as listed above.

Preferred embodiments of the steps are now described in more detail as follows:

A cryoprecipitate of human plasma is a preferred source for the fibrinogen in the method according to the invention.

The cryoprecipitate is reconstituted or solubilized under suitable buffer conditions, in particular at neutral pH, preferably in a solution buffer, subjected to adsorption in particular by Al(OH)₃, and the resulting gel is removed, preferably by centrifugation. If necessary the supernatant may be filtered. As a next step the supernatant may be virus inactivated, preferably by solvent/detergent (S/D) treatment. S/D compounds such as polysorbate 80 and TnBP (Tri-n-butyl-phosphate) are preferred.

The resulting solution may then be further purified using a chromatographic process. Typically, this can be performed by contacting the S/D treated protein solution with an anion exchange material. A preferred material for this is a material with diethylaminoethyl (DEAE) groups as anion exchange groups grafted on a matrix material. One example of such a material is Toyopeal DEAE, which uses hydroxylated methacrylic polymer beads as matrix material. The solution is contacted with the anion exchange material under conditions that fibrinogen does not bind to the weak anion exchange material. The fibrinogen is present in the flow-through.

The resulting fibrinogen solution may then be subjected to glycine precipitation, preferably by 1 to 1.5 M glycine, more preferably by about 1.2 M glycine. Preferably, additionally NaCl is used for glycine precipitation, preferably 1 to 3 M NaCl, more preferably about 2 M NaCl. The concentration may be reached by adding the glycine and/or NaCl directly to the solution. The solution is buffered in the range of 6.7 to 7.2 by 20-40 mM citrate. The fibrinogen containing precipitate can then be separated by centrifugation, preferably flow-through-centrifugation. A single precipitation of fibrinogen is usually sufficient. The fibrinogen paste might be stored at a temperature ≤−70° C.

As a next step the precipitate is resuspended and the solution may then be subjected to an UV-C treatment for further virus inactivation, especially parvovirus. In preferred embodiments, the UVivatec system (Sartorius Stedim Biotech GmbH, Germany) is used. As a buffer preferably a sodium citrate buffer is used. Preferably, the solution obtained is already suitable for the cation exchange chromatography according to the invention.

As a next step the fibrinogen solution is subjected to the cation exchange chromatography according to the above described method.

The resulting fibrinogen solution may then be further concentrated by ultrafiltration, preferably to concentrations of 20 to 70 g/l, preferably 55±10 g/l. The resulting concentrate may be diluted to the final desired concentration using a corresponding buffer. The final desired concentration is preferably in a range between 25 to 40 g/l, preferably 33±3 g/l. Further ingredients for the final drug product may be added during these steps. The pH of the solution may be adjusted, e.g. to pH 7.0±0.5.

The resulting solution may be filtrated (0.2 μm) into different vials and lyophilized. Then a final dry heat treatment (e.g. 100° C. 30 min, autoclave) may be performed as a further virus inactivating step. In the final solution or lyophilisate, the total protein content is essentially formed by fibrinogen.

The resulting lyophilisate may be reconstituted to obtain a solution comprising preferably 12 to 25 g/l fibrinogen, more preferably 18 to 24 g/l fibrinogen, 20 to 65 mmol/l arginine, more preferably 25 to 55 mmol/l arginine, 2 to 10 mmol/l citrate, more preferably 3 to 7 mmol/l citrate, and pH of 6.5 to 7.5.

The subject matter of the present invention is also a fibrinogen preparation obtainable according to the manufacturing process of the invention. This fibrinogen preparation is characterized by an advantageous purity and activity profile. In particular, the fibrinogen preparation is characterized by a very low factor XIII content. Preferably, the fibrinogen preparation has a FXIII concentration of 0.5-2.0 FXIII:Ag (% of norm) and/or a FXIII activity of less than 16 FXIII:Ac (% of norm) and thereby shows an improved purity in comparison to prior art fibrinogen products. This correspond to a value of FXIII:Ac of <0.008 IU/mg fibrinogen (at 20 mg/ml fibrinogen). 5-2.0 FXIII:Ag (% of norm) approximately correspond to 0.0003-0.0010 IU/mg fibrinogen (at 20 mg/ml fibrinogen).

By this less concomitant factors are administered, when using the fibrinogen preparation of the present invention for medical purposes. Moreover, the fibrinogen preparation shows a good or even improved clot firmness in comparison to prior art fibrinogen products. Moreover, the fibrinogen preparation shows no detectable (<0.17 mg/l) content of D-dimer which is a further evidence towards an especially advantageous physiological activity of the inventive fibrinogen preparation and also an improved purity feature.

The invention also relates to a fibrinogen product with a FXIII concentration of 0.5-2.0 FXIII:Ag (% of norm) and/or a FXIII activity of less than 16 FXIII:Ac (% of norm), and/or no detectable content of D-dimer (<0.17 mg/l) and/or a maximum clot firmness of 20 to 30 mm (Fib-tem assay at 2.5 g/L fibrinogen).

A further subject matter of the present invention is a pharmaceutical composition obtained from the fibrinogen preparation. Preferably, the pharmaceutical composition comprises a fibrinogen preparation obtainable by the inventive method as described and preferably at least one pharmaceutical carrier.

The pharmaceutical composition can be filled into suitable vials.

The pharmaceutical composition obtained shows good physiological properties as shown by a clot firmness assay and high stability. It has a lowered content of high molecular substances and is of excellent purity. The pharmaceutical composition is preferably provided as a lyophilisate.

When the pharmaceutical composition is provided as a solution, preferably from the lyophilized fibrinogen product, the content of vWF is preferably below 0.5 U/mg total protein, more preferably below 0.4 U/mg total protein. E.g. in preferred embodiments the content of vWF is below 7 U/ml at an amount of fibrinogen of 20 g/l which corresponds to 0.35 U/mg total protein.

In a preferred embodiment, the pharmaceutical composition comprises the following ingredients, when prepared as solution, preferably from the lyophilisate: fibrinogen in a concentration of 12 to 25 g/l, pH of 6.5 to 7.5, arginine 20 to 65 mmol/l, citrate 2 to 10 mmol/l.

The pharmaceutical composition obtained by the inventive method is preferably suitable for intravenous use, preferably in human.

The pharmaceutical composition is especially useful for the treatment of haemostatic disorders and/or the prevention or treatment of bleeding. In a preferred embodiment, the haemostatic disorders are congenital fibrinogen deficiency, acquired fibrinogen deficiency, traumatic injuries and the prevention or treatment of bleeding. The prevention or treatment of bleeding by administration of the pharmaceutical composition according to the invention may be done during or after surgery, especially during spinal surgery or during gynecological surgery. Thus, the invention also refers to a method of treatment of haemostatic disorders by administration of the pharmaceutical composition of the present invention.

### EXAMPLES

### Example 1

A cryoprecipitate of human plasma is used as source for the fibrinogen. The cryoprecipitate is obtained by thawing frozen plasma at 0-4° C. and separation of the precipitate.

Per kg of cryoprecipitate a mixture of 2.91 kg of water (WFI), 114 g ethanol 25% (v/v) and 9,000 IU heparin is prepared. The cryoprecipitate is added to the WFI/ethanol/heparin solution under stirring. The pH value is adjusted to 7.0.

108 g of a 2% aluminium hydroxide suspension are added per kg of cryoprecipitate used, and the mixture is stirred at 22.5° C. The pH value is adjusted to 6.55 and subsequently centrifuged by continuously operating centrifuges.

1% Polysorbate 80 and 0.3% Tri-n-butyl phosphate are added while stirring. The protein solution is stirred at 25.0° C. over a period of at least 8 hours.

The anion-exchange gel Toyopearl TSK DEAE-650 (hydroxylated methacrylic polymer beads as matrix material with diethylaminoethyl groups) is used for further purification by column chromatography. The protein loading is about 50±10 mg of protein/ml anion exchange gel.

The chloride content of the protein solution is adjusted to 120 mmol/l by addition of NaCl solution. The protein solution is applied to the column and the flow through fraction is collected.

The resulting fibrinogen solution containing 10 mM tri-sodium-citrate, 120 mM NaCl, 120 mM Glycine, 1 mM CaCl₂, 0.1% Polysorbat 80 and 0.3% TnBP, pH 7.0-7.1 is subjected to glycine precipitation. To precipitate fibrinogen, glycine is added to a final concentration of 1.2 M. NaCl is added to a final concentration of 2 M. The fibrinogen containing precipitate is then separated by centrifugation. The fibrinogen paste might be stored at a temperature ≤−70° C.

The precipitate is resuspended in a buffer (15 mM tri-sodium citrate dihydrate, pH value: 6.9+/−0.1, conductivity: 3.3+/−0.5 mS/cm). The composition beside the other proteins (e.g. 0.7-0.9 U/mg vWF) comprises TnBP, Polysorbate 80, glycine and NaCl. The composition is filtered and subjected to an UV-C treatment for virus inactivation using devices such as the UVivatec device (Sartorius Stedim Biotech). The irradiation is preferably performed at 254 nm±1 nm using 125-200 J/m².

For the following cation exchange chromatography step the column (POROS™ 50 HS) is equilibrated with equilibration buffer (15 mM tri-sodium citrate dehydrate, 65 mM sodium chloride, pH value: 6.5+/−0.1, conductivity: 9.0+/−1.5 mS/cm, 2-5 column volumes).

The liquid phase containing fibrinogen resulting from the UV irradiation step is prepared by adjusting the composition to 15 mM tri-sodium citrate, pH value: 6.5+/−0.1 and conductivity: 9.0+/−1.5 mS/cm. The column is loaded with 10-20 g/l protein per liter gel volume.

The column is rinsed with wash buffer (15 mM tri-sodium citrate dehydrate, 65 mM sodium chloride, pH value: 6.5+/−0.1, conductivity: 9.0+/−1.0 mS/cm, 2-5 column volumes).

Then the fibrinogen is eluted using elution buffer (7.5 mM tri-sodium citrate dehydrate, 150 mM sodium chloride, 75 mM L-arginine monohydrochloride, pH value: 7.0+/−0.1, conductivity: 19.5+/−1.5 mS/cm). In this step the fibrinogen is eluted from the column. Most of the vWF still binds on the column.

The column is then rinsed with a buffer with higher salt concentration (15 mM tri-sodium citrate dehydrate; 1.5 M sodium chloride, pH value: 6.5+/−0.1, conductivity: 113.5+/−5.0 mS/cm). vWF is eluted from the column. The column is then cleaned using 1 M sodium hydroxide.

In this method Albumin and IgG do not bind to the CEX material. More than 50% of the vWF present in the liquid phase containing fibrinogen can be removed by using this method.

For the production of a drug substance the eluted fractions are concentrated using ultrafiltration and the protein concentration is adjusted to 33 g fibrinogen per liter by using citrate buffer. Further ingredients may be added to form the final drug substance.

The drug substance is filtrated (0.2 μm) into different vials and lyophilized. Then a final heat treatment in a steam autoclave (100° C., 30 min) was performed as a further virus inactivating step. The product is surprisingly stable.

The final product shows a good clot stability measured as maximum clot firmness (MCF) (Fib-tem assay at 2.5 g/L fibrinogen, 25 mm) compared to a standard human plasma control (27 mm) and a plasma pool (22 mm). This is strong evidence towards a good physiological activity.

Furthermore, the fibrinogen preparation has a FXIII concentration of 1.5 FXIII:Ag (% of norm) and a FXIII activity of less than 16 FXIII:Ac (% of norm). Moreover, the fibrinogen preparation shows no detectable content of D-Dimer. Both parameters are an indication of very good purity features of a fibrinogen product according to the invention. The data is shown in table 1 (Sample 1, 2, 3: preparations according to the method of the invention according to example 1—solubilised final product, 20 mg protein/ml).

TABLE 1

| | vWF: Ag | vWF: Ag | vWF: RiCo | D-Dimer | FXIII: Ag | FXIII: Ac |
|---|---|---|---|---|---|---|
| Sample | [U/ml] | [U/mg]* | [U/ml] | [mg/l] | [% of norm] | [% of norm] |
| Sample 1 | 5.0 | 0.25 | <0.1 | <0.17 | 0.9 | <15.1 |
| Sample 2 | 6.5 | 0.33 | 0.38 | <0.17 | 1.5 | <15.1 |
| Sample 3 | 5.1 | 0.26 | 0.15 | <0.17 | 1.1 | <15.1 |

*for 20 mg/ml fibrinogen

Commercially available fibrinogen product samples contain a much higher FXIII activity and higher D-Dimer content compared to the inventive product (data not shown).

### Example 2

25 g of a glycine precipitate prepared as described in Example 1 was treated with UV-C and purified using the cation exchange chromatography (POROS™ 50 HS) as described in Example 1. Table 2 shows the composition of the loading solution, flow-through and eluate.

TABLE 2

| | | Protein | | | |
|---|---|---|---|---|---|
| | Volume | concentration | vWF:Ag | vWF:Ag | Fib:Ag |
| Sample | [ml] | [g/l] | [U/ml] | [U/mg] | [g/l] |
| Loading | 38.4 | 7.98 | 3.8 | 0.45 | 9.26 |
| solution | | | | | |
| Flow- | 45.8 | 0.0484 | 0.4 | 8.26 | 0.0104 |
| through | | | | | |
| Eluate | 55.3 | 5.29 | 1.0 | 0.19 | 6.45 |

Given that total protein essentially corresponds to fibrinogen, based on the protein concentration measured with OD 280 the yield of protein in the eluate is 95.5%. 37.9% of the total vWF is present in the fibrinogen eluate. Measured as Fib:Ag 100.3% of the fibrinogen is in the eluate.

The depletion factor of vWF in this example is 2.4 (0.45 to 0.19 U/mg), because the vWF content in the load of this concrete example is relatively low. It has been demonstrated by the inventors, that in other examples the depletion factor of vWF is in the range of 3 to 6 when the vWF content in the load material is in the range of 0.6 to 0.9 U/mg total protein.

### Example 3

A similar process as described in Example 1 with respect to the cation exchange chromatography step was performed except that Macro-Prep® High S was used as a strong cation exchange column material instead of POROS™ 50 HS. For an improved binding of the fibrinogen to the column during loading and washing the conductivity was set to 4.5 mS/cm and the pH was set to 6.0, which is above the pl of fibrinogen. Under elution conditions of pH 7 and conductivity of 19.5 mS/cm 94.8% of the fibrinogen is eluted from the column. The content of vWF in the eluted fractions is below 0.4 U/mg protein. Only around 18.3% of the vWF in the protein load of the CEX column could be found in the eluate. No polysorbate 80 or TnBP could be detected in the eluted fractions.

### Example 4

For testing the ability of the cation exchange chromatography step in reducing the content of vWF in the eluted fibrinogen fraction the cation exchange chromatography with loading material enriched in vWF (spiked) was performed using POROS™ 50 HS as described in Example 1. The vWF content of the load was 1.09 U/mg total protein (non-spiked material) up to 4.49 U/mg total protein in several spiking samples (see table 3). After selective elution of fibrinogen (150 mM NaCl, pH 7.0) vWF was eluted with the high-salt fraction (1.5 M NaCl, pH 6.5). As can be taken from table 3 the content of vWF in the eluted fibrinogen fraction (Fibrinogen Eluate) remains essentially constant with increasing vWF content of the load varying between 0.21 U/mg total protein (non-spiked material) and 0.35 U/mg total protein (spiked with 4.49 U/mg total protein in the load). The depletion factor for vWF increased with spiking of the load material starting from 5 for the non-spiked load material up to 12 obtained for the spiked load material with 4.49 U/mg total protein.

Table 4 shows depletion factors compared to the dissolved cryoprecipitate for a typical sample on larger scale. Over the total process a depletion factor of 24.4 is achieved compared to the dissolved cryoprecipitate.

TABLE 3

| | Load | | | Fibrinogen Eluate | | | |
|---|---|---|---|---|---|---|---|
| | vWF | vWF per | | vWF | vWF per | | |
| | measured | protein | Protein | measured | protein | Protein | Depletion |
| Exp. | (U/ml) | (U/mg) | (mg/ml) | (U/ml) | (U/mg) | (mg/ml) | Factor vWF |
| 1 | 6.5 | 1.09 | 5.94 | 1.2 | 0.21 | 5.71 | 5 |
| 2 | 9.9 | 1.70 | 5.84 | 1.7 | 0.30 | 5.68 | 5 |
| 3 | 12.6 | 2.14 | 5.89 | 1.8 | 0.32 | 5.62 | 6 |
| 4 | 14.6 | 2.49 | 5.87 | 1.8 | 0.32 | 5.58 | 7 |
| 5 | 17.5 | 2.99 | 5.85 | 1.4 | 0.25 | 5.56 | 11 |
| 6 | 25.8 | 4.49 | 5.75 | 1.9 | 0.35 | 5.41 | 12 |

TABLE 4

| | vWF depletion factor compared dissolved |
|---|---|
| Step | cryoprecipitate |
| Cryoprecipitate | — |
| Anion exchange | 1.3 |
| chromatography | |
| Dissolved glycine | 6.4 |
| precipitate | |
| Drug product | 24.4 |

### Example 5

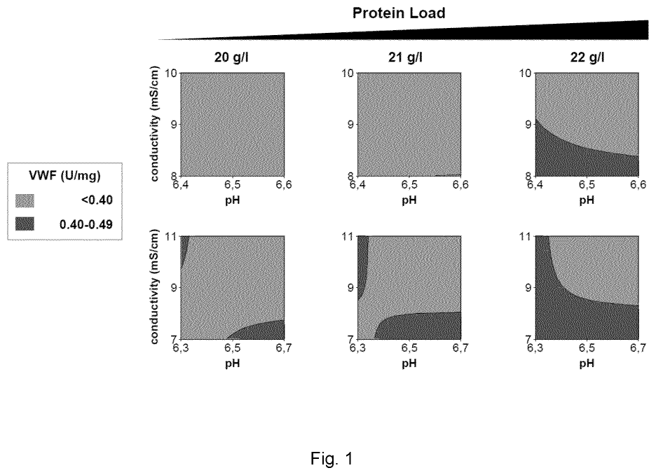

In order to demonstrate the robustness of the cation exchange chromatography step in view of the pH value and the conductivity when loading the column (POROS™ 50 HS) a range of pH 6.3 up to 6.7 was tested when loading the column according to Example 1. The conductivity when loading the column was between 7 and 11 mS/cm. The protein load was between 20 g and 22 g protein per liter column material. In all tested conditions vWF was depleted below 0.5 U/mg total protein as can be taken from FIG. 1 (FIG. 1 shows the relation of amount of vWF for different values of conductivity and pH of the loading solution for different protein loads (upper row for pH 6.4-6.6 and 8-10 mS/cm; lower row pH 6.3-6.7 and 7-11 mS/cm). The results show, that a range of 6.4 to 6.6 is especially advantageous for reduction of vWF in the fibrinogen eluate, wherein vWF is depleted below 0.4 U/mg. Moreover, the results show, that a range of conductivity of 8 to 10 mS/cm is especially advantageous for reduction of vWF in the fibrinogen eluate, resulting in a depletion of vWF below 0.4 U/mg. Moreover, it is advantageous in view of an effective depletion of vWF, that the total protein load is not more than 21 g/l, especially not more than 20 g/l column material.

### Example 6

For testing the ability of the cation exchange chromatography step to reduce the content of Polysorbat 80 and TnBP in the eluted fibrinogen fraction the cation exchange chromatography with loading material enriched in both Polysorbat 80 and TnBP (spiked) was performed using POROS™ 50 HS as described in Example 1, with the exception that the UV-C irradiation step was omitted. The Polysorbat 80 and TnBP content of the load was 1.11 mg/mL and approx. 20 μg/mL, respectively (non-spiked material) and was raised up to 10.74 mg/mL and 2590 μg/mL, respectively, in several spiking samples (see table 5). After selective elution of fibrinogen (150 mM NaCl, pH 7.0) vWF was eluted with the high-salt fraction (1.5 M NaCl, pH 6.5). The yield of fibrinogen in the column eluate remained constant over all experiments, as was deduced from chromatograms (data not shown). As can be taken from table 5 the content of Polysorbat 80 and TnBP in the eluted fibrinogen fraction (Fibrinogen Eluate) is below the limit of detection of the analytical methods for all applied spikes of Polysorbat 80/TnBP, with the exception of the highest spike of TnBP where approx. 30 μg/mL TnBP were found in the eluate. Hence Polysorbat 80 and TnBP could be depleted by up to factor 1000 using POROS™ 50 HS-chromatography on fibrinogen-containing samples. At the same time, the level of vWF found in the fibrinogen eluate remained mostly unaffected, with only a slight increase observed for the highest spikes of Polysorbat 80/TnBP.

TABLE 5

| | Load | | Fibrinogen Eluate | | |
|---|---|---|---|---|---|
| | Polysorbat | | Polysorbat | | |
| | 80 | TnBP | 80 | TnBP | |
| Experi- | measured | measured | measured | measured | vWF |
| ment | (mg/mL) | (μg/mL) | (mg/mL) | (μg/mL) | (U/mL) |
| 1 | 1.11 | approx. 20 | <0.01 | <0.8 | 0.9 |
| 2 | 1.78 | approx. 620 | <0.01 | <0.8 | 0.8 |
| 3 | 6.26 | 1230 | <0.01 | <0.8 | 0.9 |
| 4 | 10.74 | 2590 | <0.01 | approx. 30 | 1.1 |

### Example 7

The inventors were able to demonstrate that prions are effectively removed to below the limit of detection by the cation exchange chromatography step. To show this effect hamster prions (strain 263K) as a well-established test model for variant Creutzfeldt-Jacob disease were tested. The removal capacity was analyzed by prion-spiked test material. The prion titers were determined by Western blot. In the eluate of the cation exchange chromatography step prions are removed from the fibrinogen by ≥3.27 log₁₀ to below the limit of detection demonstrating the reliable removal of prions by the cation exchange chromatography step. The prions were stripped from the column by 1.5 M NaCl (high salt).

Table 6 summarizes the results of the experiment. The test material was spiked with prions (Hamster adapted scrapie isolate, strain 263K, supplier ViruSure GmbH, Austria) and samples were taken from the prion stock and spiked test material to determine the prion titers by Western blot. A volume of approximately 94 ml of spiked test material was loaded on the column and the column was washed. The flow through and wash was collected and the volume determined. The fibrinogen was eluted from the column in a volume of 50 ml and the column was washed with 1.5 M NaCl. Of each fraction, a sample was taken for prion titer analysis. The results of this study are listed in the following table 6, wherein prions are removed from the fibrinogen by 3.27 log₁₀ to below the limit of detection.

TABLE 6

| | | Endpoint Titer (log₁₀) | | | | |
|---|---|---|---|---|---|---|
| | | | Actual | | | |
| | | Determined | Titer for | | Total | |
| | | by Western | Undiluted | Volume | Titer | Reduction |
| Sample | Treatment | Blot | Sample | (mL) | (log₁₀)^{a} | (log₁₀)^{a} |
| 11.00a | Prion | 4.0 | 4.0 | 1.8 | 4.3 | |
| | spike | | | | | |
| 11.0a | spiked, | 2.0 | 2.0 | 93.9 | 4.0 | |
| | untreated | | | | | |
| 11.11 | Flow | 0.0 | 0.0 | 163.9 | ≤2.2 | ≥1.8 |
| | through | | | | | |
| | and wash | | | | | |
| 11.21 | Eluate | 0.0^{b} | <−1.0 | 50.0 | ≤0.7 | ≥3.3 |
| 11.31 | Strip | 0.5^{c} | 1.0 | 70.0 | 2.8 | 1.1 |

^{a}rounded after calculation to one decimal place
^{b}10-fold concentrated sample (−1.0log) was non-reactive
^{c}Sample was diluted

Analytical Methods

Protein Determination

Protein determination is performed by the UV absorption method (Spektralphotometer Genesys™ 6, Spektralphotometer Genesys™ 10). Proteins in solution adsorb UV light at a wavelength of 280 nm due to the presence of aromatic amino acids, mainly tyrosine and tryptophan. This property is the basis of the protein determination at 280 nm. The accuracy of the UV spectroscopic determination of protein can be decreased by the scattering of light by the test specimen. For the compensation of this effect the absorption at 360 nm is subtracted from the absorption at 280 nm.

Measurement of Fibrinogen (Fib:Ag)

The Fib:Ag concentration is determined by nephelometry at the BN Prospec (Siemens) Nephelometer. Fibrinogen forms a complex with a specific antibody. This complex causes a dispersion of irradiated light. The increased dispersion is correlated to a fibrinogen concentration.

Measurement of Activity of Fibrinogen by Clottable Protein

For the assay of fibrinogen activity (=clottable protein), the sample preparation is mixed with a suitable buffer solution containing sufficient thrombin and incubated at 37° C. The residual protein is determined in the supernatant by UV spectrometry at 280/360 nm and the result is subsequently subtracted from the total protein content (see above) to calculate the clottable protein.

Determination of Specific Activity of Fibrinogen

The specific activity of fibrinogen is determined by clottable protein activity related to total protein as measured by UV absorption (280 nm).

Measurement of Clot Stability

The clot stability is measured using the Fib-tem assay with the ROTEM whole blood analyzer (Tem Innovations GmbH, Munich). This is an established viscoelastic method for hemostasis testing in whole blood.

The test is performed according to the manufacturer's instruction. The validity of the method is tested with control preparations (Rotrol N and P).

For the measurement of Fib-tem Fibrinogen samples are dissolved according to the manufacturer's instructions and further dilutions are performed in Fibrinogen deficient plasma to obtain Fibrinogen concentrations of 1.5 g/l, 2.0 g/l and 2.5 g/l.

Measurement of the vWF Activity (vWF:Aq)

The testkit “vWF Ag” contains reagents for the immunoturbidimetric determination of von Willebrand factor antigen (vWF:Ag) in human plasma or plasma products measured with the Behring coagulation system (BCS XP).

The test is performed according to the manufacturer's instruction including the reagents provided as well as the predefined test definition and measurement instruction of the coagulation system BCS XP. The results of the samples are evaluated with a standard/reference curve.

For the preparation of the standard/reference curve Standard Human Plasma (Siemens) is used in different dilution steps in duplicate determination. The coagulation system (BCS XP) automatically dilutes the calibrator in the range of 10-200% of the norm. The validity of the reference curve is tested with the control preparation (Control Plasma N).

For the measurement a dilution series of fibrinogen concentrate samples at dilutions of 11:1, 1:10 in Owren's Veronal Buffer is prepared. All other dilutions, incubations, usage of different reagents provided in the reagent kit are automatically prepared by the test system (BCS XP).

Measurement of the Activity of FXIII (FXIII:Ac)

The activity of FXIII is determined with the photometric test (Berichrom FXIII, Siemens Healthcare Diagnostics GmbH) measured with the Behring coagulation system (BCS XP, Siemens Healthcare).

The test is performed according to the manufacturer's instruction including the reagents provided as well as the predefined test definition and measurement instruction of the coagulation system BCS XP. The samples are evaluated with a standard/reference curve.

For preparation of the calibration/reference curve, Standard Human Plasma (Siemens) is used in different dilution steps in duplicate determination. The coagulation system (BCS XP) automatically dilutes the calibrator in the range of 15-130% of the norm. The validity of the reference curve is tested with the control preparation (Control Plasma N). As used herein 100% of the norm (Siemens Standard Human Plasma (CoA)) correspond to 1 IU/ml according to WHO Standard.

For the measurement a dilution series of fibrinogen concentrate samples at dilutions of 1:1, 1:3 and 1:5 in NaCl solution (0.9%, w/v) is prepared. All other dilutions, incubations, usage of different reagents provided in the test kit are automatically prepared by the coagulation system (BCS XP).

Measurement of the Concentration of FXIII:Aq

The measurement is based on a Sandwich Elisa assay using a Matched-Pair Antibody set and VisuLize Buffer Pak (both Affinity Biologicals). The test is performed according to the manufacture's instruction including the reagents provided as well as the predefined test definition and measurement instruction. For calibration purposes a Standard Human Plasma (Siemens), 1:100 over seven steps in a 1:2 dilution, is used.

An affinity-purified polyclonal antibody to FXIII A subunit is coated in a microtitre plate. The remaining binding sites are blocked with bovine serum albumin. After washing standard and samples are applied. The bound FXIII is detected with a peroxidase conjugated antibody to FXIII. The peroxidase activity is processed with OPD (o-Phenylenediamine) and stopped with H2504. The OD is measured at 490 nm. As used herein 100% of the norm correspond approximately to 1 IU/ml according to WHO Standard.

Measurement of D-Dimer

INNOVANCE D-Dimer is a particle-enhanced, immunoturbidimetric assay for the quantitative determination of cross-linked fibrin degradation products (D-dimers) in human plasma or plasma products measured with the Behring coagulation system (BCS XP).

The test is performed according to the manufacture's instruction including the reagents provided as well as the predefined test definition and measurement instruction of the coagulation system BCS XP. The samples are evaluated with a standard/reference curve.

For the preparation of the standard/reference curve, INNOVANCE D-dimer Calibrator is used in different dilution steps in duplicate determination. The coagulation system (BCS XP) automatically dilutes the calibrator in the range of 0.17-4.4 mg/l. The validity of the reference curve is tested with the control preparation (Control Plasma N).

For the measurement a dilution series of fibrinogen concentrate samples at dilutions of 1:1, 1:5 in diluent (provided in the test kit) is prepared. All other dilutions, incubations, usage of different reagents provided in the test kit are automatically prepared by the test system (BCS XP).

Measurement of TnBP Concentration

N-Hexan is used for the extraction of TNBP from the sample solutions. Reference solutions with amounts identical to the specification limits are prepared and subjected to the same extraction procedure as other samples. The hexanoic phase from reference and sample solutions are analyzed by gas chromatography.

The evaluation of the conformity to the specification is carried out with a comparison of the heights of TNBP peaks in the chromatograms from the samples and the corresponding reference solution. The reference solutions are prepared from a valid standard solution from the TNBP-Assay-Test be dilution.

Measurement of Polysorbat 80 Concentration

The determination of Polysorbate 80 is performed by photometry according to the Ph. Eur., current edition, 2.2.25.

Polysorbate 80 in protein solutions is determined by a photometric assay. Polyoxylated compounds like Polysorbate 80 are forming a blue colored complex with ammonium cobalt thiocyanate.

Interferences due to high protein content are avoided by a deproteination with ethanol. After precipitation of protein with ethanol, the supernatant is evaporated to near dryness. The complex is extracted by dichloromethane followed by a photometric measurement at 620 nm. The calibration function is prepared without the deprotonation step.

Ristocetin Cofactor Activity (RCoF, vWF:RiCo)

The cofactor activity of von Willebrand factor is measured with BC von Willebrand Reagent, which is an in-vitro test for the determination of Ristocetin cofactor activity of von Willebrand factor in human plasma or plasma products through platelet agglutination measured with the Behring coagulation system (BCS XP).

The test is in principle performed according to the manufacture's instruction including the reagents provided. The reference curve is modified from 20-150% to 5-100% of the norm. The test definition and measurement instruction of the coagulation system BCS XP is adapted accordingly. The samples are evaluated with a standard/reference curve.

For the preparation of the calibration/reference curve Standard Human Plasma is used in different dilution steps in duplicate determination. The coagulation system (BCS XP) automatically dilutes the calibrator in the range of 5-100% of the norm. The validity of the reference curve is tested with the control preparation (Control Plasma N).

For the measurement a dilution series of fibrinogen concentrate samples at dilutions of 1:1 and 1:5 in NaCl solution (0.9%, w/v) is prepared. All other dilutions, incubations, usage of different reagents provided in the test kit are automatically prepared by the test system (BCS XP).

1. Method for manufacturing a fibrinogen preparation from a fibrinogen containing source derived from blood plasma comprising:
Providing a liquid phase containing plasmatic fibrinogen;
Contacting the liquid phase with a cation exchange chromatography material under conditions resulting in binding of fibrinogen, wherein the liquid phase has a pH in the range of 5.6 to 7.0;
Optionally washing unbound compounds from the cation exchange chromatography material; and
Eluting the fibrinogen from the cation exchange material using an elution buffer.

2. Method according to claim 1, comprising reducing an amount of von-Willebrand-factor, when the source contains von-Willebrand-factor.

3. Method according to claim 1, comprising reducing an amount of prions.

4. Method according to claim 1, wherein the liquid phase of said contacting has a pH in the range of 6.3 to 6.9.

5. Method according to claim 1, wherein the liquid phase of said contacting has an ionic strength of 5 to 15 mS/cm.

6. Method according to claim 1, wherein the cation exchange chromatography material is a strong cation exchange chromatography material.

7. Method according to claim 1, wherein the cation exchange chromatography material is a macroporous material.

8. Method according to claim 1, wherein the cation exchange chromatography material is a material comprising sulfonate functional groups.

9. Method according to claim 8, wherein the cation exchange chromatography material comprises a resin backbone consisting of crosslinked polystyrenedivinylbenzene, wherein the sulfonate functional groups are linked as sulfopropyl via a polyhydroxyl surface.

10. Method according to claim 1, wherein the washing is performed using a wash buffer with a pH in the range of 5.6 to 7.0 and an ionic strength of 5 to 15 mS/cm.

11. Method according to claim 1, wherein the elution is performed using an elution buffer with a pH of at least 0.2 units above the conditions resulting in binding of fibrinogen.

12. Method according to claim 11, wherein the elution is performed using an elution buffer with an ionic strength at least 2 mS/cm higher than the conditions resulting in binding of fibrinogen.

13. Method according to claim 1, wherein the elution buffer comprises one or more drug formulation compounds.

14. Method according to claim 13, wherein the drug formulation compound is at least one amino acid.

15. Method according to claim 14, wherein the method further comprises formulating the fibrinogen into a pharmaceutical composition.

16. Method according to claim 1, wherein the manufacturing method further comprises at least one of:
Using cryoprecipitate of human plasma as starting material;
Al(OH)3 adsorption;
S/D treatment; anion exchange chromatography and using the flow-through; glycine precipitation;
UV-C treatment;
ultrafiltration;
lyophilisation; or
heat treatment.

17. Method according to claim 16, wherein the cation exchange chromatography is performed between UV-C treatment and ultrafiltration.

18. Fibrinogen preparation obtained by the method of claim 1.

19. Fibrinogen preparation according to claim 18, wherein the fibrinogen preparation has a FXIII concentration of 0.5-2.0 FXIII:Ag (% of norm) and/or a FXIII activity of less than 16 FXIII:Ac (% of norm).

20. Fibrinogen preparation according to claim 18, wherein the fibrinogen preparation shows no detectable content of D-dimer.

21. Pharmaceutical composition obtainable from the fibrinogen preparation according to claim 18.

22. Pharmaceutical composition according to claim 21, which is a lyophilisate.

23. (canceled)

24. A method for the treatment of a haemostatic disorder or bleeding comprising administering the pharmaceutical composition according to claim 21.
